SEQUENCE LISTING (32 sequences)

SEQ ID NO: 1 - DNA, 52 bp, Homo sapiens
  misc_feature (27)..(27): n is a or g
ccagatctgacgaatgtgatggccacntcccggaaatatgaagacctgttat

SEQ ID NO: 2 - DNA, 52 bp, Homo sapiens
  misc_feature (27)..(27): n is c or t
tcaaggcaacactgcccgaggctgacngagagcgaggtgccatcatgggcat

SEQ ID NO: 3 - DNA, 52 bp, Homo sapiens
  misc_feature (27)..(27): n is a or g
gcagcgccttcttgctggcacccaatngaagccatgcgccggaccacgacgt

SEQ ID NO: 4 - DNA, 52 bp, Homo sapiens
  misc_feature (27)..(27): n is a or g
ggcagcaaaagtaatgcaatactcacntttctcttcagctgtatgaagtaaa

SEQ ID NO: 5 - DNA, 51 bp, Homo sapiens
  misc_feature (26)..(26): n is c or t
tcctcatcaccagccacgcagcccanggtcatgatgaaggggtgacctggg

SEQ ID NO: 6 - DNA, 52 bp, Homo sapiens
  misc_feature (26)..(26): n is a or c
gggaggtccagccctcatcccgcccncattccctgggcaggtggggtggcgg

SEQ ID NO: 7 - DNA, 52 bp, Homo sapiens
  misc_feature (27)..(27): n is c or t
cccgccccacgctctgtccacacccangcgccccgggagcggggccatgcct

SEQ ID NO: 8 - DNA, 52 bp, Homo sapiens
  misc_feature (27)..(27): n is a or g
cgttcactccagagctggctgaggtcnacgaagctaattttggtgccttggt

SEQ ID NO: 9 - DNA, 52 bp, Homo sapiens
  misc_feature (27)..(27): n is c or t
agcttttgtcatttttgtcatatagangaaacaactgaggcacaaaaatgtg

SEQ ID NO: 10 - DNA, 52 bp, Homo sapiens
  misc_feature (26)..(26): n is c or t
tctcaaatatatccatagttgggccnttactactttattgtattgtatttta

SEQ ID NO: 11 - DNA, 52 bp, Homo sapiens
  misc_feature (27)..(27): n is c or g
atgaccagctgttcgtgttctatgatnatgagagtcgccgtgtggagccccg

SEQ ID NO: 12 - DNA, 52 bp, Homo sapiens
  misc_feature (27)..(27): n is c or t
agttacgttccttcgatcagttgtcancattagaaagcagttccgcaagccc

SEQ ID NO: 13 - DNA, 52 bp, Homo sapiens
  misc_feature (26)..(26): n is c or g
aattttacacgaggggtgaccatctncacggtcattattgcaggagctcagc

SEQ ID NO: 14 - DNA, 52 bp, Homo sapiens
  misc_feature (27)..(27): n is a or g
cctgaaccagcaaagagaaaagaaggnccccagaaatcacaggtgggcacgt

SEQ ID NO: 15 - DNA, 52 bp, Homo sapiens
  misc_feature (27)..(27): n is c or g
cacttttccccctagttgtgtcttgcnatgctaaaggacgtcacattgcaca

SEQ ID NO: 16 - DNA, 52 bp, Homo sapiens
  misc_feature (27)..(27): n is g or t
ccagtaaatccatattgccatgaccgnagctacagggccgcttaacaaacct

SEQ ID NO: 17 - DNA, 52 bp, Homo sapiens
  misc_feature (26)..(26): n is c or t
ctaacaaactgtttcacatctttttngaggtcgtagtagttttctagatatt

SEQ ID NO: 18 - DNA, 52 bp, Homo sapiens
  misc_feature (27)..(27): n is c or g
ctttgggggctacagagaagcaggggnccagggtgggggggccttcttcagc

SEQ ID NO: 19 - DNA, 52 bp, Homo sapiens
  misc_feature (27)..(27): n is g or t
cccctgctgctgcaggccccagatgancccccagaactcttccttctgcccc

SEQ ID NO: 20 - DNA, 52 bp, Homo sapiens
  misc_feature (27)..(27): n is a or g
agatccaacatagaataggagagagtncccaaaatgatggtgaagggagacc

SEQ ID NO: 21 - DNA, 52 bp, Homo sapiens
  misc_feature (27)..(27): n is c or g
aacacttgaagcttgatatctagtttngattcaaaagcttcatttcccatat

SEQ ID NO: 22 - DNA, 52 bp, Homo sapiens
  misc_feature (26)..(26): n is c or t
cctctgcccaggctgatgggaaccancctgtagaggtccatctgcgttcaga

SEQ ID NO: 23 - DNA, 52 bp, Homo sapiens
  misc_feature (27)..(27): n is c or g
aaactctgggagattctcctattgacncagaaagcgattccttcactgatac

SEQ ID NO: 24 - DNA, 52 bp, Homo sapiens
  misc_feature (26)..(26): n is c or t
ctgaaatcactgtccctcagttcacnggtcttgtctgcttcgtcgtcaaaaa

SEQ ID NO: 25 - DNA, 52 bp, Homo sapiens
  misc_feature (27)..(27): n is c or g
aggcggacagcacccaagacaagaagnctcccatgatgcagtctcagagccg

SEQ ID NO: 26 - DNA, 52 bp, Homo sapiens
  misc_feature (27)..(27): n is a or c
acatgcaggcgatggtgcaactcatangctacatgcacacctactgcctccc

SEQ ID NO: 27 - DNA, 52 bp, Homo sapiens
  misc_feature (27)..(27): n is c or g
tctccgaagcatgtggggcgtgctgantgccctgggaaggtctggagcagag

SEQ ID NO: 28 - DNA, 52 bp, Homo sapiens
  misc_feature (27)..(27): n is a or g
ggaggcaataggttttgaggggcatgnggacggggttcagcctccagggtcc

SEQ ID NO: 29 - DNA, 52 bp, Homo sapiens
  misc_feature (26)..(26): n is a or g
catcacaccgcggtactgggcgctgnctgtagcgcgcactggcccctgactt

SEQ ID NO: 30 - DNA, 52 bp, Homo sapiens
  misc_feature (26)..(26): n is a or g
ggcttggcactggtcttatacacacnggctgacctgaaaccttatcctagag

SEQ ID NO: 31 - DNA, 52 bp, Homo sapiens
  misc_feature (27)..(27): n is c or t
tggcctgcttgctgttcttacaggganggaggcaatggcggccagcacttcc

SEQ ID NO: 32 - DNA, 52 bp, Homo sapiens
  misc_feature (27)..(27): n is c or g
gcgcgcgggcgtgcgagcagcgaaagngacaggggcaaagtgagtgacctgc

(19) United States
(12) Patent Application Publication
(10) Pub. No.: US 2013/0130918 A1
(43) Pub. Date: May 23, 2013

(54) EXERCISE GENOTYPING

(71) Applicants: Graeme John Smith, Victoria (AU); Nick Argyrou, Victoria (AU); Helen Argyrou, Victoria (AU); Harry Banaharis, Victoria (AU)

(72) Inventors: Graeme John Smith, Victoria (AU); Nick Argyrou, Victoria (AU); Helen Argyrou, Victoria (AU); Harry Banaharis, Victoria (AU)

(73) Assignee: Genetics Investment Pty. Ltd.

(21) Appl. No.: 13/577,193

(22) PCT Filed: Feb. 4, 2011
(86) PCT No.: PCT/AU2011/000114

§ 371 Date: Jan. 25, 2013

(30) Foreign Application Priority Data
Feb. 5, 2010 (AU) 2010900472

(51) Int. Cl. C12Q 1/68

(52) U.S. Cl. CPC C12Q 1/6876
USPC 506/2; 435/6.11

(57) ABSTRACT

The invention relates to a method for identifying a genetic predisposition of a subject to increased exercise endurance, increased muscular power, muscle damage and/or injury risk, a method for formulating an exercise program for improving physical performance, a kit suitable for use in the methods and use of the method for improving physical performance.

### FIELD

The invention relates to methods for identifying a subject's genetic predisposition to improved physical performance in different exercise activities.

### BACKGROUND

There is a limit to each individual's capacity to perform exercise which depends on the nature of the task and a variety of other factors including genetics.

Human physical activity patterns have been established by natural selection over millions of years, with the adaptive pressures inherent in environmental niches exerting a defining influence on the human genetic profile of individuals that evolved there. Although precise attribution between athletic nature and nurture are not possible, it is a generally accepted sport science proposition that genetic variations account for approximately 50% of athletic variation in performance, with 50% attributable to both the individual athlete's response to training, as well as social factors, such as the support provided to the individual in pursuit of their goals. Certain body types are well suited to particular types of athletic functions and movements. The Rift Valley of Africa has produced more world-champion and Olympic-champion distance runners than any other place on Earth, due to the slender, relatively long-striding people of that district, who live at altitudes in excess of 6,562 ft (2,000 m). These physical attributes have created a superlative human form for distance running. In contrast, the people who live near the Baltic Sea in northeast Europe often possess tall, lean, muscular frames, ideally suited to sports such as basketball. These two examples are based on an observation of the athletic success that these groups have enjoyed in the stated sports. Genetics research seeks to uncover the scientific foundation in support of these observations.

WO/2004/024947 discloses methods of selecting or matching a sport or sporting event to an individual (e.g. a sprint/power sport or an endurance sport) and predicting elite athletic performance in these sports, the methods involving assessing ACTN3 genotype. However, a weakness of current exercise performance predisposition identification strategies is that they are either entirely based on observation of performance data and physical measurements, or only investigate a limited number of genes, thus their predictive accuracy and value is very limited. It is an aim of an embodiment to address at least one of the limitations of the prior art. It is to be understood that if any prior art publication is referred to herein such reference does not constitute an admission that the publication forms a part of the common general knowledge in the art in Australia or any other country.

### SUMMARY

A first aspect provides a method for determining a genetic predisposition of a subject to an exercise performance trait, the method comprising:
a) assaying a genetic sample from the subject for a plurality of polymorphisms associated with one or more exercise performance traits selected from exercise endurance, muscular power, muscle damage and/or injury risk, to obtain a polymorphism profile;
b) analysing the polymorphism profile to identify predisposition alleles;
c) assigning a polymorphism score to each polymorphism tested based on the predisposition allele for the associated trait;
d) calculating a total genotype score for each trait, based on a combination of the polymorphism scores for each trait; and
e) classifying the subject's endurance predisposition, power predisposition, muscle damage predisposition and/or injury risk predisposition based on the total genotype score for the one or more exercise performance traits.

The subject's predisposition for each trait may be classified as being low, medium or high predisposition.

Individual polymorphisms associated with exercise endurance, muscular power, muscle damage and/or injury risk will be known to the person of skill in the art. Such polymorphisms include polymorphisms associated with exercise endurance selected from polymorphisms in genes selected from the group consisting of ACE, ACTN3, ADBR2, AMPD1, CKM, EPAS1, GDF-8, HFE, NFATC4, NOS3, NRF2, PPARα, PPARδ, PPARγC1α, PPARγC1β, TFAM, UCP2, UCP3 and VEGFA, but not the combination of genes ACTN3 with ACE when the assaying is for a plurality of polymorphisms associated with exercise endurance only; polymorphisms associated with muscular power selected from polymorphisms in genes selected from the group consisting of ACE, ACTN3, AMPD1, COL1α1, HIF-1α, IL6, PPARα, PPARγ and VDR; polymorphisms associated with muscle damage selected from polymorphisms in genes selected from the group consisting of IGF2, IL6, IGF2AS and TNFα; and polymorphisms associated with injury risk selected from polymorphisms in genes selected from the group consisting of COL1α1, COL5α1 and MMP3. However, a person of skill in the art would appreciate that the invention also encompasses other polymorphisms associated with each of these traits that may have already been identified, or may be identified in the future.

The polymorphism may be a single nucleotide polymorphism (SNP).

In embodiments of the first aspect, the method comprises assaying two of the traits, or three of the traits, or all four of the traits.

The method may comprise the step of assaying the genetic sample to determine a haplogroup. The step of assaying the genetic sample to determine a haplogroup may comprise assaying a mitochondrial polymorphism or a Y-chromosome polymorphism.

The identification of a subject's genetic predisposition to an exercise performance trait allows the provision of an exercise program taking into account this predisposition to allow the subject to achieve improved physical performance, for example, improving health, well being and/or body composition, improving athletic performance, and/or avoiding injury.

Accordingly, a second aspect provides a method for formulating an exercise program for improving physical performance in a subject, the method comprising:
a) assaying a genetic sample from the subject for a plurality of polymorphisms associated with one or more exercise performance traits selected from exercise endurance, muscular power, muscle damage and/or injury risk, to obtain a polymorphism profile;
b) analysing the polymorphism profile to identify predisposition alleles;
c) assigning a polymorphism score to each polymorphism tested based on the predisposition allele for the associated trait;
d) calculating a total genotype score for each trait, based on a combination of the polymorphism scores for each trait;
e) classifying the subject's endurance predisposition, power predisposition, muscle damage predisposition and/or injury risk predisposition based on the total genotype score for the one or more exercise performance traits; and
f) formulating an exercise plan for the subject, comprising exercises suitable for the subject's predisposition.

The method of the second aspect provides an integrated approach to improving physical performance by accounting for both the genetic predisposition of the subject and the most appropriate exercise program for the subject's genetic profile.

A third aspect provides a kit, comprising a sampler for obtaining a genetic sample from a subject, when the genetic sample is assayed according to the method of the first or second aspect.

A fourth aspect provides a kit for identifying a genetic predisposition of a subject to exercise endurance, muscular power, muscle damage and/or injury risk, the kit comprising a sampler for obtaining a genetic sample from a subject and reagent for assaying a genetic sample obtained from the subject for a plurality of polymorphisms in genes selected from the group consisting of ACE, ACTN3, ADBR2, AMPD1, CKM, COL1α1, COL5α1, EPAS1, GDF-8, HFE, HIF-1α, IGF2, IL6, IGF2AS, MMP3, NFATC4, NOS3, NRF2, PPARα, PPARδ, PPARγC1α, PPARγC1β, PPARγ, TFAM, TNFα, UCP2, UCP3, VDR and VEGFA or combination thereof.

A fifth aspect provides use of a method according to the first or second aspects, or a kit according to the third or fourth aspects, for improving physical performance.

### DETAILED DESCRIPTION

Analysis of a genetic sample from a subject, with regard to polymorphisms associated with one or more traits selected from exercise endurance, muscular power, muscle damage and/or injury risk, provides information that may be used to classify an individual with regard to their genetic predisposition to an exercise performance trait. The classification of an individual's genetic predisposition can be used to select an exercise program comprising appropriate levels and frequencies of power and endurance exercise activities for the subject's predisposition and this should lead to improved physical performance and reduced rates of injury.

As used herein, “genetic predisposition” refers to the presence of genetic polymorphisms in a genetic sample that are associated with phenotypic traits. A phenotypic trait is an observable trait that is conferred by the presence of one or more genes or genetic polymorphisms.

As used herein, “exercise performance trait” refers to a phenotypic trait associated with exercise performance. As used herein, “exercise performance” refers to attaining improved physical performance from engaging in particular types and frequencies of exercise. As used herein, “physical performance” refers to the physical fitness of the subject. This encompasses cardiovascular fitness, body composition fitness and the overall condition of subject's musculature as well as athletic ability. “Body composition” as used herein refers to the percentages of fat, muscle, water and bone in a subject's body. “Athletic ability” as used herein refers to the success level of performing physical tasks e.g. running fast, jumping high, running for a long time, lifting heavy weights. For example, an exercise performance trait may be exercise endurance, muscular power, muscle damage and/or injury risk. As used herein, “exercise endurance” refers to the phenotypic trait of being predisposed to improved physical performance in endurance exercise activities. This predisposition may be identified by the presence of one or more polymorphisms associated with endurance characteristics. As used herein, “muscular power” refers to the phenotypic trait of being predisposed to improved physical performance in power exercise activities. This predisposition may be identified by the presence of one or more polymorphisms associated with power characteristics. As used herein, “muscle damage” refers to the phenotypic trait of being predisposed to muscle damage. This predisposition may be identified by the presence of one or more polymorphisms associated with muscle damage. As used herein, “injury risk” refers to the phenotypic trait of being predisposed to injury risk. Injury risk includes tendon, joint and/or ligament injury. This predisposition may be identified by the presence of one or more polymorphisms associated with injury risk.

Whilst a subject may consider that they are aware of their ideal exercise plan for improved physical performance, the present method provides a systematic approach, with scientific validation, to identifying specific exercise programs or types of exercise programs that should result in improved physical performance. Moreover, the present methods enable inappropriate exercise programs to be substituted with more appropriate exercise programs for any subject. Indeed, the exercise program may be formulated to adjust the ratio of strength or power activities in a personalised manner.

As used herein, “exercise” refers to physical exertion for the sake of improvement of physical performance. Exercise may be aerobic or anaerobic, and the amount or ratio of each may be related to the polymorphism profile and predisposition of a subject to a particular exercise performance trait. As used herein, “exercise plan”, or “exercise program”, refers to a specific set of physical exertions that are to be carried out for a specified duration and at a specified frequency.

As used herein, “polymorphism profile” refers to the combination of polymorphisms possessed by a subject with regard to the parts of the genome assessed. A subject's polymorphism profile, comprising one or more genotypes, may be used to differentiate between subjects that are likely to exhibit different responses to a particular stimulus, in this instance, to the type and frequency of exercises.

As used herein, “gene” refers to any genetic material that provides instructions for the organism to perform some biological structure of function. Most commonly, but not exclusively, a gene will comprise one or more exons encoding the amino acid sequence of a polypeptide or protein, intervening introns, and non-coding regions including the promoter, 5′-untranslated region and the 3′-untranslated region. That is, a gene specifically included non-coding regions. The term “gene” also includes portions such as enhancer elements that may function in trans with the coding portion of a gene.

“Genome” or “genomic” as used herein refers to the complete genetic material encoding an organism. The term “genotype” refers to the fundamental biochemical composition of the genetic material of a subject organism, and implicitly refers to the differences in that composition between individuals. Accordingly, the term “genotyping” refers to the act of assaying to determine the composition of the genetic material of a subject organism, often for comparison to the genotype of another individual. A genotype is usually determined from a polymorphism.

A “polymorphism” refers to the existence of two or more forms or variations in the DNA of a particular gene that has a frequency of at least 1% in the population. In the context of a genotype, it refers to the existence of two or more forms of a genotype, which differ in their nucleotide composition. A polymorphism includes a restriction fragment length polymorphism (RFLP), a tandem repeat, a variable number tandem repeat (VNTR), a short tandem repeat (STR), a minisatellite, a microsatellite, a simple sequence length polymorphism (SSLP), an insertion-deletion (indel), an amplified fragment length polymorphism (AFLP), a random amplification of polymorphic DNA (RAPD), a single nucleotide polymorphism (SNP), and any other genetic feature that may be distinguished between individuals. In one embodiment, the polymorphism may be a SNP. Polymorphisms exist in at least two states or alleles.

“Single nucleotide polymorphism” or “SNP” as used herein means an alteration of a single nucleotide at a defined position within the genome of at least two individuals of the same species. SNPs usually comprise two alternative nucleotides, for example A or T, or, C or G. Such a SNP may be used to predict a subject's suitability to particular exercises.

“Allele” as used herein refers to one of the two copies of a genetic unit contained within a subject's genome. In a population, more then two alleles may exist. However, any subject will usually only possess a subset of alleles present in the population. For example, a mammalian subject will possess two alleles for a particular gene, although the population may comprise three or more alleles.

A “predisposition allele” refers to the specific allele of a genotype that confers a higher predisposition for a specific characteristic. To determine a subject's predisposition for exercise endurance (i.e. the subject's endurance predisposition), muscular power (i.e. the subject's power predisposition), muscular damage (i.e. the subject's muscle damage predisposition) and/or injury risk (i.e. the subject's injury risk predisposition), a polymorphism score may be given to the genotype results to quantify the influence of an individual polymorphism on the subject's genetic predisposition to a particular exercise performance trait. Polymorphism scores range from 0—no predisposition, to 1—intermediate predisposition, to 2—high predisposition. As used herein, the term “polymorphism score” refers to a numerical value allocated to the predisposition alleles associated with a given characteristic. A polymorphism score may be modified based on the subject's haplotype, haplogroup or ancestral origin, for example as determined using a mitochondrial polymorphism or a Y-chromosome polymorphism. Because ancestry plays a role in genetic adaptation to exercise, genotyping may include analysis of maternal and paternal haplogroups to further determine exercise performance predisposition. As used herein, a “haplotype” refers to a specific combination of alleles at two or more genetic loci that are transmitted together. In turn, a “haplogroup”, is a collection of similar haplotypes and relates to genetic populations and ancestral origin. A haplogroup may be predicted from a haplotype. In one embodiment, a haplogroup comprises a mitochondrial polymorphism or haplogroup, which is maternal, or a Y-chromosome polymorphism or haplogroup, which is paternal.

A total genotype scoring system, adapted from the scoring system used by Williams and Folland (2008), may be used to calculate an overall total genotype score. The total genotype score quantifies the combined influence of the predisposition alleles possessed by a subject on their genetic predisposition to a particular exercise performance trait. A total genotype score may be between 0 and 100, with a total genotype score of 100 representing the highest possible predisposition to the exercise performance trait, and a total genotype score of 0 representing the lowest possible predisposition to the exercise performance trait.

The total genotype score may be calculated by adding together all of the individual polymorphism scores for a given trait and multiplying the sum of the individual polymorphism scores by 100 divided by the number of individual polymorphisms multiplied by 2, which is the highest possible polymorphism score for a given genotype The formula is shown below:

TGS=(100/16)×(Gene Score₁+Gene Score₂+ . . . Gene Score₈)

NOTE: a total of 8 polymorphisms are included in this calculation (8×2=16).

To examine the distribution of total genotype scores in the general population, a hypothetical dataset of 1,000 individuals, with a randomly generated genotype based on the population frequency of each genotype, may be used. An individual may then be compared to the average genotype score of this hypothetical dataset and assigned a classification with regard to their predisposition (e.g. low predisposition, moderate predisposition, high predisposition) based on their total genotype score in comparison to the average total genotype score generated from the hypothetical dataset.

For example, a subject possessing a score classifying them as having an endurance predisposition may experience improved physical performance when they engage in endurance exercise activities. Exercise endurance is associated with several physical factors, including one's ability to utilize oxygen, anaerobic threshold/lactate threshold, and muscle efficiency. Higher peaks in VO₂ max have been correlated with an enhanced ability to convert the bodily fuel sources into energy and an increased predisposition to exercise endurance. Likewise, a higher proportion of Type I muscle fibres has been correlated with an increased predisposition to exercise endurance. Type I muscles fibres are one of two types of muscle fibres. Type I muscle fibres produce more energy at lower intensities hence, they are very well suited to endurance exercise activities where the exercise needs to be maintained over longer durations. Type I muscle fibres are not very well suited to short bursts of explosive energy because they rely on oxygen availability. Hence, there is a trade-off between power and endurance. Endurance exercise activities include exercises done at low intensity but high repetition and typically cause muscle fatigue only after a significant period of time. Typical endurance exercise activities include long-distance running and long-distance swimming.

A subject possessing a score classifying them as having a power predisposition may experience improved physical performance when they engage in power exercise activities. Muscular strength and power are associated with the size of the muscle and the intensity or frequency at which the force is generated. Like exercise endurance, researchers have found that there are a number of genetic variations associated with muscular power and strength. Muscle is made up of bundles of fibres (Type I and Type II) that work together to lift objects including our own body parts. The Type II muscle fibres are a lot bigger and stronger than the Type I fibres so they are more suited to lifting heavier objects and moving more weight. Consequently, a higher percentage of Type II muscle fibres has been correlated with muscular power. Power training works muscle groups using weights heavy enough to result in substantial fatigue within 8 to 12 repetitions. How heavy those weights are will depend on the subject and how strong they are and the weight will increase as the subject trains and improves their overall physical condition. However, the improvement in physical performance does not change the subject's predisposition to that particular type of exercise activity. Power exercise activities include exercises done at high intensity but low repetition and typically cause significant muscle fatigue in a short period of time. Typical power exercise activities include tennis, volleyball, gymnastics, fencing and golf swing.

A subject may be classified as having a medium or moderate predisposition to exercise endurance and/or muscular power. This means that the subject has some alleles associated with muscular power as well as some alleles associated with exercise endurance. These subjects are suited to activities that fall in the middle range and utilise both Type I and Type II muscle fibres. Typical moderate power/moderate endurance exercise activities include rowing, skiing, swimming, hockey, soccer and basketball. The endurance impact of the activity may be increased or decreased by doing the activity for more or less time respectively. The power impact of the activity may be increased or decreased by exerting more or less force when doing the activity respectively.

Injury prevention and a subject's ability to recover from exercise are very important. A subject's risk of injury is associated with the environment in which they exercise or compete and intrinsic factors such as their genetic makeup. Research has shown that certain genetic variants, such as on the TNF-α and IGF2 genes, have been associated with increased level of muscle damage and muscle soreness following high-intensity weight training explaining why some subjects would potentially require extended recovery periods after exercise to allow for full muscle repair. A subject possessing a score classifying them as having a muscle damage predisposition may experience improved physical performance when they engage in less frequent exercise activities and are likely to benefit from spending a little more time recovering from a hard-workout, especially work-outs at high-intensity such as eccentric and plyometric training. This is very important because continuing to train when the body is not fully recovered may result in over-training and injury. Less frequent exercise activities allow for a longer period of recovery and healing following each workout.

A subject possessing a score classifying them as having an injury risk predisposition may experience improved physical performance when they engage in stretching and flexibility exercises as well as training to improve technique so as to prevent injury. Several genetic variants may affect the main structural component of tendons, ligaments and joints. For example, genetic variants in the COL5α1 gene have been shown to modify the risk of Achilles tendon injuries.

The more predisposition alleles for each respective trait that the subject possesses, the greater their predisposition is for that trait. The probability of having all polymorphisms in any one of the traits is the multiplication of the frequency of the predisposition allele in the population, across all polymorphisms in the respective category. For example, hypothetically—If the population frequency of the predisposition alleles A, B, and C was 10%, 15%, and 40% respectively then the probability of having predisposition alleles A, B & C is 10%×15%×40%, which is 0.6%.

Polymorphisms associated with exercise endurance include the following SNPs:

ACE (encoding angiotensin I converting enzyme (peptidyl-dipeptidase A) 1) NCBI unique identifier RS4343 which is located on chromosome 17 of the Homo sapiens genome and comprises the following sequence (SEQ ID NO: 1), or its reverse complement: CCAGATCTGACGAATGTGATGGCCAC[A/G]TCCCGGAAATATGAAGACCTGTTAT The A/A allele, or its reverse complement, confers the highest predisposition to exercise endurance.

ACTN3 (encoding actinin, alpha 3) NCBI unique identifier RS1815739 which is located on chromosome 11 of the Homo sapiens genome and comprises the following sequence (SEQ ID NO: 2), or its reverse complement: TCAAGGCAACACTGCCCGAGGCTGAC[C/T]GAGAGCGAGGTGCCATCATGGGCAT The T/T allele, or its reverse complement, confers the highest predisposition to exercise endurance.

ADBR2 (encoding adrenergic, beta-2-, receptor, surface) NCBI unique identifier RS1042713 which is located on chromosome 5 of the Homo sapiens genome and comprises the following sequence (SEQ ID NO: 3), or its reverse complement: GCAGCGCCTTCTTGCTGGCACCCAAT[A/G]GAAGCCATGCGCCGGACCACGACGT The A/A allele, or its reverse complement, confers the highest predisposition to exercise endurance.

AMPD1 (encoding adenosine monophosphate deaminase 1 (isoform M)) NCBI unique identifier RS17602729 which is located on chromosome 1 of the Homo sapiens genome and comprises the following sequence (SEQ ID NO: 4), or its reverse complement: GGCAGCAAAAGTAATGCAATACTCAC[A/G]TTTCTCTTCAGCTGTATGAAGTAAA Where the G/G and A/G alleles, or their reverse complements, confer the highest predisposition to exercise endurance.

CKM (encoding creatine kinase, muscle) NCBI unique identifier RS1803285 which is located on chromosome 19 of the Homo sapiens genome and comprises the following sequence (SEQ ID NO: 5), or its reverse complement: TCCTCATCACCAGCCACGCAGCCCA[C/T]GGTCATGATGAAGGGGTGACCTGGG The C/T allele, or its reverse complement, confers the highest predisposition to exercise endurance.

EPAS1(1) (encoding endothelial PAS domain protein 1) NCBI unique identifier RS1867785 which is located on chromosome 2 of the Homo sapiens genome and comprises the following sequence (SEQ ID NO: 8), or its reverse complement: CGTTCACTCCAGAGCTGGCTGAGGTC[A/G]NCGAAGCTAATTTTGGTGCCTTGGT The G/G allele, or its reverse complement, confers the highest predisposition to exercise endurance.

EPAS1(2) (encoding endothelial PAS domain protein 1) NCBI unique identifier RS11689011 which is located on chromosome 2 of the Homo sapiens genome and comprises the following sequence (SEQ ID NO: 9), or its reverse complement: AGCTTTTGTCATTTTTGTCATATAGA[C/T]GAAACAACTGAGGCACAAAAATGTG The T/T allele, or its reverse complement, confers the highest predisposition to exercise endurance.

GDF-8 (MSTN; encoding myostatin) NCBI unique identifier RS1805086 which is located on chromosome 2 of the Homo sapiens genome and comprises the following sequence (SEQ ID NO: 10), or its reverse complement: TCTCAAATATATCCATAGTTGGGCC[C/T]TTACTACTTTATTGTATTGTATTTTA The T/T allele, or its reverse complement, confers the highest predisposition to exercise endurance.

HFE (encoding hemochromatosis) NCBI unique identifier RS1799945 which is located on chromosome 6 of the Homo sapiens genome and comprises the following sequence (SEQ ID NO: 11), or its reverse complement: ATGACCAGCTGTTCGTGTTCTATGAT[C/G]ATGAGAGTCGCCGTGTGGAGCCCCG The C/G allele, or its reverse complement, confers the highest predisposition to exercise endurance.

NFATC4 (encoding nuclear factor of activated T-cells, cytoplasmic, calcineurin-dependent 4) NCBI unique identifier RS2229309 which is located on chromosome 14 of the Homo sapiens genome and comprises the following sequence (SEQ ID NO: 18), or its reverse complement: CTTTGGGGGCTACAGAGAAGCAGGGG[C/G]CCAGGGTGGGGGGGCCTTCTTCAGC The G/G allele, or its reverse complement, confers the highest predisposition to exercise endurance.

NOS3 (encoding nitric oxide synthase 3 (endothelial cell)) NCBI unique identifier RS1799983 which is located on chromosome 7 of the Homo sapiens genome and comprises the following sequence (SEQ ID NO: 19), or its reverse complement: CCCCTGCTGCTGCAGGCCCCAGATGA[G/T]CCCCCAGAACTCTTCCTTCTGCCCC The G/G allele, or its reverse complement, confers the highest predisposition to exercise endurance.

NRF2 (GABPB1; encoding GA binding protein transcription factor, beta subunit 1) NCBI unique identifier RS7181866 which is located on chromosome 15 of the Homo sapiens genome and comprises the following sequence (SEQ ID NO: 20), or its reverse complement: AGATCCAACATAGAATAGGAGAGAGT[A/G]CCCAAAATGATGGTGAAGGGAGACC The G/G allele, or its reverse complement, confers the highest predisposition to exercise endurance.

PPARα (encoding peroxisome proliferator-activated receptor alpha) NCBI unique identifier RS4253778 which is located on chromosome 22 of the Homo sapiens genome and comprises the following sequence (SEQ ID NO: 21), or its reverse complement: AACACTTGAAGCTTGATATCTAGTTT[C/G]GATTCAAAAGCTTCATTTCCCATAT The G/G allele, or its reverse complement, confers the highest predisposition to exercise endurance.

PPARδ (encoding peroxisome proliferator-activated receptor delta) NCBI unique identifier RS2016520 which is located on chromosome 6 of the Homo sapiens genome and comprises the following sequence (SEQ ID NO: 22), or its reverse complement: CCTCTGCCCAGGCTGATGGGAACCA[C/T]CCTGTAGAGGTCCATCTGCGTTCAGA The C/C allele, or its reverse complement, confers the highest predisposition to exercise endurance.

PPARγC1α (encoding peroxisome proliferator-activated receptor gamma, coactivator 1 alpha) NCBI unique identifier RS8192678 which is located on chromosome 4 of the Homo sapiens genome and comprises the following sequence (SEQ ID NO: 24), or its reverse complement: CTGAAATCACTGTCCCTCAGTTCAC[C/T]GGTCTTGTCTGCTTCGTCGTCAAAAA The C/C allele, or its reverse complement, confers the highest predisposition to exercise endurance.

PPARγC1β(1) (encoding peroxisome proliferator-activated receptor gamma, coactivator 1 beta) NCBI unique identifier RS7732671 which is located on chromosome 5 of the Homo sapiens genome and comprises the following sequence (SEQ ID NO: 25), or its reverse complement: AGGCGGACAGCACCCAAGACAAGAAG[C/G]CTCCCATGATGCAGTCTCAGAGCCG The C/C allele, or its reverse complement, confers the highest predisposition to exercise endurance.

PPARγC1β(2) (encoding peroxisome proliferator-activated receptor gamma, coactivator 1 beta) NCBI unique identifier RS11959820 which is located on chromosome 5 of the Homo sapiens genome and comprises the following sequence (SEQ ID NO: 26), or its reverse complement: ACATGCAGGCGATGGTGCAACTCATA[A/C]GCTACATGCACACCTACTGCCTCCC The A/A allele, or its reverse complement, confers the highest predisposition to exercise endurance.

TFAM (encoding transcription factor A, mitochondrial) NCBI unique identifier RS1937 which is located on chromosome 10 of the Homo sapiens genome and comprises the following sequence (SEQ ID NO: 27), or its reverse complement: TCTCCGAAGCATGTGGGGCGTGCTGA[C/G]TGCCCTGGGAAGGTCTGGAGCAGAG The C/C allele, or its reverse complement, confers the highest predisposition to exercise endurance.

UCP2 (encoding uncoupling protein 2 (mitochondrial, proton carrier)) NCBI unique identifier RS660339 which is located on chromosome 11 of the Homo sapiens genome and comprises the following sequence (SEQ ID NO: 29), or its reverse complement: CATCACACCGCGGTACTGGGCGCTG[A/G]CTGTAGCGCGCACTGGCCCCTGACTT The A/A allele, or its reverse complement, confers the highest predisposition to exercise endurance.

UCP3 (encoding uncoupling protein 3 (mitochondrial, proton carrier)) NCBI unique identifier RS1800849 which is located on chromosome 11 of the Homo sapiens genome and comprises the following sequence (SEQ ID NO: 30), or its reverse complement: GGCTTGGCACTGGTCTTATACACAC[A/G]GGCTGACCTGAAACCTTATCCTAGAG The A/A allele, or its reverse complement, confers the highest predisposition to exercise endurance.

VEGFA (encoding vascular endothelial growth factor A) NCBI unique identifier RS2010963 which is located on chromosome 6 of the Homo sapiens genome and comprises the following sequence (SEQ ID NO: 32), or its reverse complement: GCGCGCGGGCGTGCGAGCAGCGAAAG[C/G]GACAGGGGCAAAGTGAGTGACCTGC The C/C allele, or its reverse complement, confers the highest predisposition to exercise endurance.

Polymorphisms associated with muscular power include the following SNPs:

ACE (encoding angiotensin I converting enzyme (peptidyl-dipeptidase A) 1) NCBI unique identifier RS4343 which is located on chromosome 17 of the Homo sapiens genome and comprises the following sequence (SEQ ID NO: 1), or its reverse complement: CCAGATCTGACGAATGTGATGGCCAC[A/G]TCCCGGAAATATGAAGACCTGTTAT The G/G allele, or its reverse complement, confers the highest predisposition to muscular power.

ACTN3 (encoding actinin, alpha 3) NCBI unique identifier RS1815739 which is located on chromosome 11 of the Homo sapiens genome and comprises the following sequence (SEQ ID NO: 2), or its reverse complement: TCAAGGCAACACTGCCCGAGGCTGAC[C/T]GAGAGCGAGGTGCCATCATGGGCAT The C/C allele, or its reverse complement, confers the highest predisposition to muscular power.

AMPD1 (encoding adenosine monophosphate deaminase 1 (isoform M)) NCBI unique identifier RS17602729 which is located on chromosome 1 of the Homo sapiens genome and comprises the following sequence (SEQ ID NO: 4), or its reverse complement: GGCAGCAAAAGTAATGCAATACTCAC[A/G]TTTCTCTTCAGCTGTATGAAGTAAA The G/G and A/G alleles, or their reverse complements, confer the highest predisposition to muscular power.

COL1α1 (encoding collagen, type I, alpha 1) NCBI unique identifier RS1800012 which is located on chromosome 17 of the Homo sapiens genome and comprises the following sequence (SEQ ID NO: 6), or its reverse complement: GGGAGGTCCAGCCCTCATCCCGCCC[A/C]CATTCCCTGGGCAGGTGGGGTGGCGG The C/C allele, or its reverse complement, confers the highest predisposition to muscular power.

HIF-1α (encoding hypoxia inducible factor 1, alpha subunit (basic helix-loop-helix transcription factor)) NCBI unique identifier RS11549465 which is located on chromosome 14 of the Homo sapiens genome and comprises the following sequence (SEQ ID NO: 12), or its reverse complement: AGTTACGTTCCTTCGATCAGTTGTCA[C/T]CATTAGAAAGCAGTTCCGCAAGCCC The C/T allele, or its reverse complement, confers the highest predisposition to muscular power.

IL6 (encoding interleukin 6 (interferon, beta 2)) NCBI unique identifier RS1800795 which is located on chromosome 7 of the Homo sapiens genome and comprises the following sequence (SEQ ID NO: 15), or its reverse complement: CACTTTTCCCCCTAGTTGTGTCTTGC[C/G]ATGCTAAAGGACGTCACATTGCACA The G/G allele, or its reverse complement, confers the highest predisposition to muscular power.

PPARα (encoding peroxisome proliferator-activated receptor alpha) NCBI unique identifier RS4253778 which is located on chromosome 22 of the Homo sapiens genome and comprises the following sequence (SEQ ID NO: 21), or its reverse complement: AACACTTGAAGCTTGATATCTAGTTT[C/G]GATTCAAAAGCTTCATTTCCCATAT The C/C allele, or its reverse complement, confers the highest predisposition to muscular power.

PPARγ (encoding peroxisome proliferator-activated receptor gamma) NCBI unique identifier RS1801282 which is located on chromosome 3 of the Homo sapiens genome and comprises the following sequence (SEQ ID NO: 23), or its reverse complement: AAACTCTGGGAGATTCTCCTATTGAC[C/G]CAGAAAGCGATTCCTTCACTGATAC The G/G and G/C alleles, or their reverse complements, confer the highest predisposition to muscular power.

VDR (encoding vitamin D (1,25-dihydroxyvitamin D3) receptor) NCBI unique identifier RS2228570 which is located on chromosome 12 of the Homo sapiens genome and comprises the following sequence (SEQ ID NO: 31), or its reverse complement: TGGCCTGCTTGCTGTTCTTACAGGGA[C/T]GGAGGCAATGGCGGCCAGCACTTCC The T/T allele, or its reverse complement, confers the highest predisposition to muscular power.

Polymorphisms associated with muscle damage include the following SNPs:

IGF2(1) (encoding insulin-like growth factor 2 (somatomedin A)) NCBI unique identifier RS3213221 which is located on chromosome 11 of the Homo sapiens genome and comprises the following sequence (SEQ ID NO: 13), or its reverse complement: AATTTTACACGAGGGGTGACCATCT[C/G]CACGGTCATTATTGCAGGAGCTCAGC The G/G allele, or its reverse complement, confers the highest predisposition to muscle damage.

IGF2(2) (encoding insulin-like growth factor 2 (somatomedin A)) NCBI unique identifier RS680 which is located on chromosome 11 of the Homo sapiens genome and comprises the following sequence (SEQ ID NO: 14), or its reverse complement: CCTGAACCAGCAAAGAGAAAAGAAGG[A/G]CCCCAGAAATCACAGGTGGGCACGT The A/A allele, or its reverse complement, confers the highest predisposition to muscle damage.

IL6 (encoding interleukin 6 (interferon, beta 2)) NCBI unique identifier RS1800795 which is located on chromosome 7 of the Homo sapiens genome and comprises the following sequence (SEQ ID NO: 15), or its reverse complement: CACTTTTCCCCCTAGTTGTGTCTTGC[C/G]ATGCTAAAGGACGTCACATTGCACA The C/C and G/C alleles, or their reverse complements, confer the highest predisposition to muscle damage.

IGF2AS (encoding IGF2AS readthrough transcript) NCBI unique identifier RS7924316 which is located on chromosome 11 of the Homo sapiens genome and comprises the following sequence (SEQ ID NO: 16), or its reverse complement: CCAGTAAATCCATATTGCCATGACCG[G/T]AGCTACAGGGCCGCTTAACAAACCT The T/T allele, or its reverse complement, confers the highest predisposition to muscle damage.

TNFα (encoding tumour necrosis factor (TNF superfamily, member 2)) NCBI unique identifier RS1800629 which is located on chromosome 6 of the Homo sapiens genome and comprises the following sequence (SEQ ID NO: 28), or its reverse complement: GGAGGCAATAGGTTTTGAGGGGCATG[A/G]GGACGGGGTTCAGCCTCCAGGGTCC The G/G allele, or its reverse complement, confers the highest predisposition to muscle damage.

Polymorphisms associated with injury risk include the following SNPs:

COL1α1 (encoding collagen, type I, alpha 1) NCBI unique identifier RS1800012 which is located on chromosome 17 of the Homo sapiens genome and comprises the following sequence (SEQ ID NO: 6), or its reverse complement: GGGAGGTCCAGCCCTCATCCCGCCC[A/C]CATTCCCTGGGCAGGTGGGGTGGCGG The C/C and NC alleles, or their reverse complements, confer the highest predisposition to injury risk.

COL5α1 (encoding collagen, type V, alpha 1) NCBI unique identifier RS12722 which is located on chromosome 9 of the Homo sapiens genome and comprises the following sequence (SEQ ID NO: 7), or its reverse complement: CCCGCCCCACGCTCTGTCCACACCCA[C/T]GCGCCCCGGGAGCGGGGCCATGCCT The C/T allele, or its reverse complement, confers the highest predisposition to injury risk.

MMP3 (encoding matrix metallopeptidase 3 (stromelysin 1, progelatinase)) NCBI unique identifier RS679620 which is located on chromosome 11 of the Homo sapiens genome and comprises the following sequence (SEQ ID NO: 17), or its reverse complement: CTAACAAACTG TTTCACATCTTTTT[C/T]GAGGTCGTAGTAGTTTTCTAGATATT The C/C allele, or its reverse complement, confers the highest predisposition to injury risk.

The assay may be performed against genes in one or more of the traits and assays may be performed simultaneously or sequentially. If more than one assay is to be performed, the assays may be performed on distinct genetic samples from the same subject, for example spatially or temporally distinct samples, or on the same sample.

The plurality of polymorphisms associated with exercise endurance may be selected from polymorphisms in genes selected from the group consisting of ACE, ACTN3, ADBR2, AMPD1, CKM, EPAS1, GDF-8, HFE, NFATC4, NOS3, NRF2, PPARα, PPARδ, PPARγC1α, PPARγC1β, TFAM, UCP2, UCP3 and VEGFA, but not the combination of genes ACTN3 with ACE when the assaying is for a plurality of polymorphisms associated with exercise endurance only.

The plurality of polymorphisms associated with muscular power exercise may be selected from polymorphisms in genes selected from the group consisting of ACE, ACTN3, AMPD1, COL1α1, HIF-1α, IL6, PPARα, PPARγ and VDR.

The plurality of polymorphisms associated with muscle damage may be selected from polymorphisms in genes selected from the group consisting of IGF2, IL6, IGF2AS and TNFα.

The plurality of polymorphisms associated with injury risk may be selected from polymorphisms in genes selected from the group consisting of COL1α1, COL5α1 and MMP3.

The method of the first aspect may comprise assaying a genetic sample from the subject for polymorphisms associated with exercise endurance for at least 2, 3, 4, 5, 6, 7, 8, 9, 10, 11, 12, 13, 14, 15, 16, 17, 18 or 19 genes selected from the group consisting of ACE, ACTN3, ADBR2, AMPD1, CKM, EPAS1, GDF-8, HFE, NFATC4, NOS3, NRF2, PPARα, PPARδ, PPARγC1α, PPARγC1β, TFAM, UCP2, UCP3 and VEGFA, but not the combination of genes ACTN3 with ACE when the assaying is for a plurality of polymorphisms associated with exercise endurance only; assaying a genetic sample from the subject for polymorphisms associated muscular power exercise for at least 2, 3, 4, 5, 6, 7, 8 or 9 genes selected from the group consisting of ACE, ACTN3, AMPD1, COL1α1, HIF-1α, IL6, PPARα, PPARγ and VDR; assaying a genetic sample from the subject for polymorphisms associated with muscle damage for at least 2, 3 or 4 genes selected from the group consisting of IGF2, IL6, IGF2AS and TNFα; and/or assaying a genetic sample from the subject for polymorphisms associated with injury risk for at least 2 or 3 genes selected from the group consisting of COL1α1, COL5α1 and MMP3.

In a certain embodiment, the polymorphisms are SNPs selected from the group consisting of SEQ ID NO: 1 (RS4343), or the reverse complement thereof, SEQ ID NO: 2 (RS1815739), or the reverse complement thereof, SEQ ID NO: 3 (RS1042713), or the reverse complement thereof, SEQ ID NO: 4 (RS17602729), or the reverse complement thereof, SEQ ID NO: 5 (RS1803285), or the reverse complement thereof, SEQ ID NO: 8 (RS1867785), or the reverse complement thereof, SEQ ID NO: 9 (RS11689011), or the reverse complement thereof, SEQ ID NO: 10 (RS1805086), or the reverse complement thereof, SEQ ID NO: 11 (RS1799945), or the reverse complement thereof, SEQ ID NO: 18 (RS2229309), or the reverse complement thereof, SEQ ID NO: 19 (RS1799983), or the reverse complement thereof, SEQ ID NO: 20 (RS7181866), or the reverse complement thereof, SEQ ID NO: 21 (RS4253778), or the reverse complement thereof, SEQ ID NO: 22 (RS2016520), or the reverse complement thereof, SEQ ID NO: 24 (RS8192678), or the reverse complement thereof, SEQ ID NO: 25 (RS7732671), or the reverse complement thereof, SEQ ID NO: 26 (RS11959820), or the reverse complement thereof, SEQ ID NO: 27 (RS1937), or the reverse complement thereof, SEQ ID NO: 29 (RS660339), or the reverse complement thereof, SEQ ID NO: 30 (RS1800849), or the reverse complement thereof, and SEQ ID NO: 32 (RS2010963), or the reverse complement thereof.

In another embodiment, the polymorphisms are SNPs selected from the group consisting of SEQ ID NO: 1 (RS4343), or the reverse complement thereof, SEQ ID NO: 2 (RS1815739), or the reverse complement thereof, SEQ ID NO: 4 (RS17602729), or the reverse complement thereof, SEQ ID NO: 6 (RS1800012), or the reverse complement thereof, SEQ ID NO: 12 (RS11549465), or the reverse complement thereof, SEQ ID NO: 15 (RS1800795), or the reverse complement thereof, SEQ ID NO: 21 (RS4253778), or the reverse complement thereof, SEQ ID NO: 23 (RS1801282), or the reverse complement thereof, and SEQ ID NO: 31 (RS2228570), or the reverse complement thereof.

In yet another embodiment, the polymorphisms are SNPs selected from the group consisting of SEQ ID NO: 13 (RS3213221), or the reverse complement thereof, SEQ ID NO: 14 (RS680), or the reverse complement thereof, SEQ ID NO: 15 (RS1800795), or the reverse complement thereof, SEQ ID NO: 16 (RS7924316), or the reverse complement thereof, and SEQ ID NO: 28 (RS1800629), or the reverse complement thereof.

In yet another embodiment, the polymorphisms are SNPs selected from the group consisting of SEQ ID NO: 6 (RS1800012), or the reverse complement thereof, SEQ ID NO: 7 (RS12722), or the reverse complement thereof, and SEQ ID NO: 17 (RS679620), or the reverse complement thereof.

In one embodiment, the predisposition allele of SEQ ID NO: 1 (RS4343) is A/A, or the reverse complement thereof, SEQ ID NO: 2 (RS1815739) is T/T, or the reverse complement thereof, SEQ ID NO: 3 (RS1042713) is A/A, or the reverse complement thereof, SEQ ID NO: 4 (RS17602729) is G/G or A/G, or the reverse complements thereof, SEQ ID NO: 5 (RS1803285) is C/T, or the reverse complement thereof, SEQ ID NO: 8 (RS1867785) is G/G, or the reverse complement thereof, SEQ ID NO: 9 (RS11689011) is T/T, or the reverse complement thereof, SEQ ID NO: 10 (RS1805086) is T/T, or the reverse complement thereof, SEQ ID NO: 11 (RS1799945) is C/G, or the reverse complement thereof, SEQ ID NO: 18 (RS2229309) is G/G, or the reverse complement thereof, SEQ ID NO: 19 (RS1799983) is G/G, or the reverse complement thereof, SEQ ID NO: 20 (RS7181866) is G/G, or the reverse complement thereof, SEQ ID NO: 21 (RS4253778) is G/G, or the reverse complement thereof, SEQ ID NO: 22 (RS2016520) is C/C, or the reverse complement thereof, SEQ ID NO: 24 (RS8192678) is C/C, or the reverse complement thereof, SEQ ID NO: 25 (RS7732671) is C/C, or the reverse complement thereof, SEQ ID NO: 26 (RS11959820) is A/A, or the reverse complement thereof, SEQ ID NO: 27 (RS1937) is C/C, or the reverse complement thereof, SEQ ID NO: 29 (RS660339) is A/A, or the reverse complement thereof, SEQ ID NO: 30 (RS1800849) is A/A, or the reverse complement thereof, and/or SEQ ID NO: 32 (RS2010963) is C/C, or the reverse complement thereof.

In another embodiment, the predisposition allele of SEQ ID NO: 1 (RS4343) is G/G, or the reverse complement thereof, SEQ ID NO: 2 (RS1815739) is C/C, or the reverse complement thereof, SEQ ID NO: 4 (RS17602729) is G/G or A/G, or the reverse complements thereof, SEQ ID NO: 6 (RS1800012) is C/C, or the reverse complement thereof, SEQ ID NO: 12 (RS11549465) is C/T, or the reverse complement thereof, SEQ ID NO: 15 (RS1800795) is G/G, or the reverse complement thereof, SEQ ID NO: 21 (RS4253778) is C/C, or the reverse complement thereof, SEQ ID NO: 23 (RS1801282) is G/G or G/C, or the reverse complements thereof, and/or SEQ ID NO: 31 (RS2228570) is T/T, or the reverse complement thereof.

In another embodiment, the predisposition allele of SEQ ID NO: 13 (RS3213221) is G/G, or the reverse complement thereof, SEQ ID NO: 14 (RS680) is A/A, or the reverse complement thereof, SEQ ID NO: 15 (RS1800795) is C/C or G/C, or the reverse complements thereof, SEQ ID NO: 16 (RS7924316) is T/T, or the reverse complement thereof, and/or SEQ ID NO: 28 (RS1800629) is G/G, or the reverse complement thereof.

In yet another embodiment, the predisposition allele of SEQ ID NO: 6 (RS1800012) is C/C or NC, or the reverse complements thereof, SEQ ID NO: 7 (RS12722) is C/T, or the reverse complement thereof, and/or SEQ ID NO: 17 (RS679620) is C/C, or the reverse complement thereof.

As the aim of genotyping is to identify if a subject is carrying gene versions that orient them to a predisposition towards particular kinds and frequencies of exercise, polymorphisms for testing may be selected from one, two, three, or four traits to determine the type of predisposition. The greater the number of polymorphisms tested the greater the likelihood of accurately identifying a genetic predisposition towards improved physical performance from engaging in particular kinds and frequencies of exercise. Genotyping may be conducted by any means known in the art. For example, genotyping may include polymerase chain reaction (PCR), RT-PCR, nucleic acid sequencing, primer extension reactions, or an array-based method. For example, genotyping may be performed using array or chip technology. A number of array technologies are known in the art and commercially available for use, including, but not limited to, static arrays (e.g. photolithographically set), suspended arrays (e.g. soluble arrays), and self assembling arrays (e.g. matrix ordered and deconvoluted).

Alternatively, a polymorphism may be detected in genetic material using techniques including direct analysis of isolated nucleic acids such as Southern blot hybridisation or direct nucleic acid sequencing. Another alternative for direct analysis of polymorphisms is the INVADER® assay (Third Wave Technologies, Inc (Madison, Wis.)). This assay is generally based upon a structure-specific nuclease activity of a variety of enzymes, which are used to cleave a target-dependent cleavage structure, thereby indicating the presence of specific nucleic acid sequences or specific variations thereof in a sample.

Conveniently, assaying a polymorphism may utilise genomic DNA. However, assaying a polymorphism may also be performed utilising mRNA or cDNA, for example. Assaying a polymorphism also encompassed indirectly assaying a genetic polymorphism by detecting a consequential difference in a gene product, for example, by detecting an amino acid substitution in cases where a polymorphism results in a codon change.

The “subject” includes a mammal. The mammal may be a human, or may be a domestic, zoo, or companion animal. While it is particularly contemplated that the method disclosed herein are suitable for humans, they are also applicable to animals, including treatment of companion animals such as dogs and cats, and domestic animals such as horses, cattle and sheep, or zoo animals such as felids, canids, bovids, and ungulates. In one embodiment, the subject may be a human. The term “subject” is used interchangeably with “individual” and “person”.

A “genetic sample” comprises any form of genetic material specific to a subject. A genetic sample may be a deoxyribonucleic acid (DNA) or a ribonucleic acid (RNA), or any modification or derivative thereof. Thus, a genetic sample usually will include a cell derived from a subject. The genetic sample may be a blood sample, a mucosal sample, a saliva sample, a hair sample including a follicle, urine, mouth wash, amniotic fluid or other tissue or fluid sample that contains a cell, DNA or RNA that is suitable for genotyping. In one embodiment, the genetic sample may be a buccal swab. A genetic sample may be obtained using a “genetic sampler”, which refers to a device for obtaining DNA or RNA suitable for genotyping. A genetic sampler may be a swab, a scraper or a container or any device capable of capturing genetic material, such as a cell, for genotype analysis. Genetic material may be isolated from the genetic sample by any method known in the art, for example extraction and precipitation or silica-based extraction. A genetic sampler may be included in a kit. A kit may also include a reagent for detecting a genotype. For example, a reagent may include a support or support material such as, without limitation, a nylon or nitrocellulose membrane, bead, or plastic film, or glass, or microarray or nanoarray, comprising a set of polymorphisms from which a subject's exercise performance predisposition may be determined. The kit may comprise other reagents necessary for performing the genotyping, including, but not limited to, labelled or unlabelled nucleic acid probes, detection label, buffers, and controls. The kit may include instructions for use.

The kit would enable determination of whether a subject is genetically predisposed to endurance exercise, power exercise, muscle damage and/or injury risk. This information may be used to screen subjects, such as athletes and amateur sports people, including children and adults, and classify them based on their genetic predisposition to particular exercise performance traits. Screening of subjects who are not yet involved in exercise programs could help to identify people who are more likely to benefit from particular kinds and frequencies of exercise. Appropriate measures may then be implemented in kinds and frequencies of exercise. Such a genetic approach will help professionals in the field of physical training to improve outcomes by providing appropriate advice based on a subject's genetic predisposition to one or more exercise performance traits.

In addition, it is contemplated that the methods disclosed herein may be used to determine a subject's genetic predisposition to an exercise performance traits and improve physical performance for subjects that may be prone to muscle atrophy such as the elderly or infirm, subjects requiring immobilisation, and/or subjects suffering from diseases including anorexia, cancer, HIV/AIDS, congestive heart disease, chronic obstructive pulmonary disease, renal failure, liver failure and severe burns.

Alternatively, or additionally, the subject's polymorphism profile may be used to recommend appropriate dietary and other lifestyle changes such as counselling to further improve the subject's physical performance and health benefits.

In order to enhance the benefit of knowing one's genetic predisposition to an exercise performance trait or to enhance the effect of observing an exercise plan formulated on the basis of that predisposition, the method may be combined with counselling and/or dietary advice. “Counselling” refers to the provision of advice, opinion or instruction with the goal of directing the conduct of a subject. As used herein, such conduct relates to choice of particular kinds and frequencies of exercise activities and in some instances compliance with a formulated exercise plan. Counselling is chiefly aimed at improving the mental well-being of a subject, whereas dietary advice is mainly aimed at improving the physical well-being of a subject. Thus, the method contemplates a holistic approach to fitness, where the benefit of observance of a genetic predisposition to an exercise performance trait or compliance with a formulated exercise program may be enhanced by supplementary activities.

As used herein, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention. It must also be noted that, as used in the subject specification, the singular forms “a”, “an” and “the” include plural aspects unless the context clearly dictates otherwise.

It will be understood to persons skilled in the art of the invention that many modifications may be made without departing from the spirit and scope of the invention.

### EXAMPLES

### Example 1

A Caucasian subject is selected for analysis and classification of their exercise performance predisposition. A genetic sample from the individual is assayed for:

a) polymorphisms associated with exercise endurance consisting of:
ACE (rs4343) and ADBR2 (rs1042713);
ACE (rs4343) and PPARα (rs4253778);
ACE (rs4343) and PPARγC1α (rs8192678);
ACE (rs4343) and PPARδ (rs2016520);
ACE (rs4343) and VEGFA (rs2010963);
ADBR2 (rs1042713) and PPARα (rs4253778);
ADBR2 (rs1042713) and PPARγC1α (rs8192678);
ADBR2 (rs1042713) and PPARδ (rs2016520);
ADBR2 (rs1042713) and VEGFA (rs2010963);
PPARα (rs4253778) and PPARγC1α (rs8192678);
PPARα (rs4253778) and PPARδ (rs2016520);
PPARα (rs4253778) and VEGFA (rs2010963);
PPARγC1α (rs8192678) and PPARδ (rs2016520);
PPARγC1α (rs8192678) and VEGFA (rs2010963);
PPARδ (rs2016520) and VEGFA (rs2010963);
ACE (rs4343), ADBR2 (rs1042713) and PPARα (rs4253778);
ACE (rs4343), ADBR2 (rs1042713) and PPARγC1α (rs8192678);
ACE (rs4343), ADBR2 (rs1042713) and PPARδ (rs2016520);
ACE (rs4343), ADBR2 (rs1042713) and VEGFA (rs2010963);
ACE (rs4343), PPARα (rs4253778) and PPARγC1α (rs8192678);
ACE (rs4343), PPARα (rs4253778) and PPARδ (rs2016520);
ACE (rs4343), PPARα (rs4253778) and VEGFA (rs2010963);
ACE (rs4343), PPARγC1α (rs8192678) and PPARδ (rs2016520);
ACE (rs4343), PPARγC1α (rs8192678) and VEGFA (rs2010963);
ACE (rs4343), PPARδ (rs2016520) and VEGFA (rs2010963);
ADBR2 (rs1042713), PPARα (rs4253778) and PPARγC1α (rs8192678);
ADBR2 (rs1042713), PPARα (rs4253778) and PPARδ (rs2016520);
ADBR2 (rs1042713), PPARα (rs4253778) and VEGFA (rs2010963);
ADBR2 (rs1042713), PPARγC1α (rs8192678) and PPARδ (rs2016520);
ADBR2 (rs1042713), PPARγC1α (rs8192678) and VEGFA (rs2010963);
ADBR2 (rs1042713), PPARδ (rs2016520) and VEGFA (rs2010963);
PPARα (rs4253778), PPARγC1α (rs8192678) and PPARδ (rs2016520);
PPARα (rs4253778), PPARγC1α (rs8192678) and VEGFA (rs2010963);
PPARα (rs4253778), PPARδ (rs2016520) and VEGFA (rs2010963);
PPARγC1α (rs8192678), PPARδ (rs2016520) and VEGFA (rs2010963);
ACE (rs4343), ADBR2 (rs1042713), PPARα (rs4253778) and PPARγC1α (rs8192678);
ACE (rs4343), ADBR2 (rs1042713), PPARα (rs4253778) and PPARδ (rs2016520);
ACE (rs4343), ADBR2 (rs1042713), PPARα (rs4253778) and VEGFA (rs2010963);
ACE (rs4343), PPARα (rs4253778), PPARγC1α (rs8192678) and PPARδ (rs2016520);
ACE (rs4343), PPARα (rs4253778), PPARγC1α (rs8192678) and VEGFA (rs2010963);
ACE (rs4343), PPARγC1α (rs8192678), PPARδ (rs2016520) and VEGFA (rs2010963);
ADBR2 (rs1042713), PPARα (rs4253778), PPARγC1α (rs8192678) and PPARδ (rs2016520);
ADBR2 (rs1042713), PPARα (rs4253778), PPARγC1α (rs8192678) and VEGFA (rs2010963);
ADBR2 (rs1042713), PPARγC1α (rs8192678), PPARδ (rs2016520) and VEGFA (rs2010963);
PPARα (rs4253778), PPARγC1α (rs8192678), PPARδ (rs2016520) and VEGFA (rs2010963);
ACE (rs4343), ADBR2 (rs1042713), PPARα (rs4253778), PPARγC1α (rs8192678) and PPARδ (rs2016520);
ACE (rs4343), ADBR2 (rs1042713), PPARα (rs4253778), PPARγC1α (rs8192678) and VEGFA (rs2010963);
ACE (rs4343), PPARα (rs4253778), PPARγC1α (rs8192678), PPARδ (rs2016520) and VEGFA (rs2010963);
ADBR2 (rs1042713), PPARα (rs4253778), PPARγC1α (rs8192678), PPARδ (rs2016520) and VEGFA (rs2010963); or
ACE (rs4343), ADBR2 (rs1042713), PPARα (rs4253778), PPARγC1α (rs8192678), PPARδ (rs2016520) and VEGFA (rs2010963);
   b) polymorphisms associated with muscular power consisting of:
ACE-(rs4343) and AMPD1-(rs17602729);
ACE-(rs4343) and PPARα-(rs4253778);
ACE-(rs4343) and VDR-(rs2228570);
ACE-(rs4343) and PPARγ-(rs1801282);
ACE-(rs4343) and HIF-1α-(rs11549465);
AMPD1-(rs17602729) and PPARα-(rs4253778);
AMPD1-(rs17602729) and VDR-(rs2228570);
AMPD1-(rs17602729) and PPARγ-(rs1801282);
AMPD1-(rs17602729) and HIF-1α-(rs11549465);
PPARα-(rs4253778) and VDR-(rs2228570);
PPARα-(rs4253778) and PPARγ-(rs1801282);
PPARα-(rs4253778) and HIF-1α-(rs11549465);
VDR-(rs2228570) and PPARγ-(rs1801282);
VDR-(rs2228570) and HIF-1α-(rs11549465);
PPARγ-(rs1801282) and HIF-1α-(rs11549465);
ACE-(rs4343), AMPD1-(rs17602729) and PPARα-(rs4253778);
ACE-(rs4343), AMPD1-(rs17602729) and VDR-(rs2228570);
ACE-(rs4343), AMPD1-(rs17602729) and PPARγ-(rs1801282);
ACE-(rs4343), AMPD1-(rs17602729) and HIF-1α-(rs11549465);
ACE-(rs4343), PPARα-(rs4253778) and VDR-(rs2228570);
ACE-(rs4343), PPARα-(rs4253778) and PPARγ-(rs1801282);
ACE-(rs4343), PPARα-(rs4253778) and HIF-1α-(rs11549465);
ACE-(rs4343), VDR-(rs2228570) and PPARγ-(rs1801282);
ACE-(rs4343), VDR-(rs2228570) and HIF-1α-(rs11549465);
ACE-(rs4343), PPARγ-(rs1801282) and HIF-1α-(rs11549465);
AMPD1-(rs17602729), PPARα-(rs4253778) and VDR-(rs2228570);
AMPD1-(rs17602729), PPARα-(rs4253778) and PPARγ-(rs1801282);
AMPD1-(rs17602729), PPARα-(rs4253778) and HIF-1α-(rs11549465);
AMPD1-(rs17602729), VDR-(rs2228570) and PPARγ-(rs1801282);
AMPD1-(rs17602729), VDR-(rs2228570) and HIF-1α-(rs11549465);
AMPD1-(rs17602729), PPARγ-(rs1801282) and HIF-1α-(rs11549465);
PPARα-(rs4253778), VDR-(rs2228570) and PPARγ-(rs1801282);
PPARα-(rs4253778), VDR-(rs2228570) and HIF-1α-(rs11549465);
PPARα-(rs4253778), PPARγ-(rs1801282) and HIF-1α-(rs11549465);
VDR-(rs2228570), PPARγ-(rs1801282) and HIF-1α-(rs11549465);
ACE-(rs4343), AMPD1-(rs17602729), PPARα-(rs4253778) and VDR-(rs2228570);
ACE-(rs4343), AMPD1-(rs17602729), PPARα-(rs4253778) and PPARγ—(rs1801282);
ACE-(rs4343), AMPD1-(rs17602729), PPARα-(rs4253778) and HIF-1α-(rs11549465);
ACE-(rs4343), PPARα-(rs4253778), VDR-(rs2228570) and PPARγ-(rs1801282);
ACE-(rs4343), PPARα-(rs4253778), VDR-(rs2228570) and HIF-1α-(rs11549465);
ACE-(rs4343), VDR-(rs2228570), PPARγ-(rs1801282) and HIF-1α-(rs11549465);
AMPD1-(rs17602729), PPARα-(rs4253778), VDR-(rs2228570) and PPARγ—(rs1801282);
AMPD1-(rs17602729), PPARα-(rs4253778), VDR-(rs2228570) and HIF-1α—(rs11549465);
AMPD1-(rs17602729), VDR-(rs2228570), PPARγ-(rs1801282) and HIF-1α—(rs11549465);
PPARα-(rs4253778), VDR-(rs2228570), PPARγ-(rs1801282) and HIF-1α-(rs11549465);
ACE-(rs4343), AMPD1-(rs17602729), PPARα-(rs4253778), VDR-(rs2228570) and PPARγ-(rs1801282);
ACE-(rs4343), AMPD1-(rs17602729), PPARα-(rs4253778), VDR-(rs2228570) and HIF-1α-(rs11549465);
ACE-(rs4343), PPARα-(rs4253778), VDR-(rs2228570), PPARγ-(rs1801282) and HIF-1α-(rs11549465);
AMPD1-(rs17602729), PPARα-(rs4253778), VDR-(rs2228570), PPARγ—(rs1801282) and HIF-1α-(rs11549465); or
ACE-(rs4343), AMPD1-(rs17602729), PPARα-(rs4253778), VDR-(rs2228570), PPARγ-(rs1801282) and HIF-1α-(rs11549465)
   c) polymorphisms associated with muscle damage consisting of:
IGF2 (rs680) and IGF2AS (rs7924316);
IGF2 (rs680) and IL6 (rs1800795);
IGF2 (rs680) and TNFα (rs1800629);
IGF2AS (rs7924316) and IL6 (rs1800795);
IGF2AS (rs7924316) and TNFα (rs1800629);
IL6 (rs1800795) and TNFα (rs1800629)
IGF2 (rs680), IGF2AS (rs7924316) and IL6 (rs1800795);
IGF2 (rs680), IGF2AS (rs7924316) and TNFα (rs1800629);
IGF2 (rs680), IL6 (rs1800795) and TNFα (rs1800629);
IGF2AS (rs7924316), IL6 (rs1800795) and TNFα (rs1800629); or
IGF2 (rs680), IGF2AS (rs7924316), IL6 (rs1800795) and TNFα (rs1800629); and/or
   d) polymorphisms associated with injury risk consisting of:
COL1α1 (rs1800012) and COL5α1 (rs12722)

For each category, the subject may obtain the following results for the forward strand (in square brackets are the allele frequencies for Caucasians for that SNP, followed by the OR for the improved physical performance and beneficial health effects alleles for a Caucasian population):

Exercise Endurance Polymorphisms

| | |
|---|---|
| ACE - rs4343 | (AA) - [GG = 0, GA = 1, AA = 2] |
| ADBR2 - rs1042713 | (AA) - [GG = 0, GA = 1, AA = 2] |
| PPARα - rs4253778 | (GG) - [CC = 0, GC = 1, GG = 2] |
| PPARγC1α - rs8192678 | (CC) - [TT = 0, CT = 1, CC = 2] |
| PPARδ - rs2016520 | (CC) - [TT = 0, CT = 1, CC = 2] |
| VEGFA - rs2010963 | (CG) = [GG = 0, CG = 1, CC = 2] |

Muscular Power Polymorphisms

| | |
|---|---|
| ACE - rs4343 | (AA) - [AA = 0, GA = 1, GG = 2] |
| AMPD1 - rs17602729 | (GG) - [GG = 0, GA = 0, AA = 2] |
| PPARα - rs4253778 | (CC) - [CC = 0, CT = 1, TT = 2] |
| VDR - rs2228570 | (CT) - [CC = 0, CT = 1, TT = 2] |
| PPARγ - rs1801282 | (CC) - [CC = 0, CG = 2, GG = 2] |
| HIF-1α - rs11549465 | (CC) - [CC = 0, CT = 2, TT = 0] |

Muscle Damage Polymorphisms

| | |
|---|---|
| IGF2 - rs680 | (GG) - [GG = 0, AG = 2, AA = 2] |
| IGF2AS - rs7924316 | (CC) - [CC = 0, CT = 2, TT = 2] |
| IL6 - rs1800795 | (CC) - [CC = 0, CG = 2, GG = 2] |
| TNFα - rs1800629 | (TT) - [TT = 0, GT = 1, GG = 2] |

Injury Risk Polymorphisms

| | |
|---|---|
| COL1α1 - rs1800012 | (AA) - [AA = 0, AC = 2, CC = 2] |
| COL5α1 - rs12722 | (CC) - [CC = 0, CT = 2, TT = 0] |

The relative predisposition is calculated for each SNP according to the method mentioned in the scoring methodology and then summed to produce a combined score followed by converting the combined score into a total genotype score as explained in the methodology adapted from Williams and Folland (2008) using the allele frequencies found in the general population. Individuals are then compared to the average genotype score in the general population and assigned into a classification as explained in the scoring methodology.

Endurance Polymorphisms

| | |
|---|---|
| ACE - rs4343 (AA) - | 2 |
| ADBR2 - rs1042713 (AA) - | 2 |
| PPARα - rs4253778 (GG) - | 2 |
| PPARγC1α - rs8192678 (CC) - | 2 |
| PPARδ - rs2016520 (CC) - | 2 |
| VEGFA - rs2010963 (CG) - | 1 |
| Combined score | 11 |
| Total genotype Score | 68.8 |
| Classification | HIGH endurance |

Power Polymorphisms

| | |
|---|---|
| ACE - rs4343 (AA) - | 0 |
| AMPD1 - rs17602729 (GG) - | 0 |
| PPARα - rs4253778 (CC) - | 0 |
| VDR - rs2228570 (CT) - | 1 |
| PPARγ - rs1801282 (CC) - | 0 |
| HIF-1α - rs11549465 (CC) - | 0 |
| Combined score | 1 |
| Total genotype Score | 8.3 |
| Classification | LOW power |

Muscle Damage Polymorphisms

| | |
|---|---|
| IGF2 - rs680 (GG) - | 0 |
| IGF2AS - rs7924316 (CC) - | 0 |
| IL6 - rs1800795 (CC) - | 0 |
| TNFα - rs1800629 (TT) - | 0 |
| Combined score | 0 |
| Total genotype Score | 0 |
| Classification | LOW muscle damage |

Injury risk polymorphisms

| | |
|---|---|
| COL1α1 - rs1800012 (AA) - | 0 |
| COL5α1 - rs12722 (CC) - | 0 |
| Combined score | 0 |
| Total genotype Score | 0 |
| Classification | LOW injury risk |

### Example 2

Subjects were selected for analysis and classification of their exercise performance predisposition. Prior to analysis and classification, these subjects reported:

1) poor training results,

2) fatigue and poor recovery following exercise, and

3) injuries associated with training

After having their exercise performance predisposition analysed and classified, the subjects modified their training programs based on their exercise performance predisposition. Individuals modified their training according to their exercise performance predisposition for a period of 4 to 6 months. Individuals who modified their training reported

1) an increase in training ability in endurance, and/or power

2) greater muscle recovery following exercise and training results, and

3) increased awareness about potential injury risks Based on the above observations, a training program that is modified and given to an individual based on their exercise performance predisposition, may achieve benefits in terms of training response and muscle recovery following exercise, as well as benefits associated with an increased awareness about injury risks. As such, maintenance of a training program matched to an individual's exercise performance predisposition, in the long term, is expected to improve training results, improve muscle recovery, and importantly, increase an individual's awareness about potential injury risk and consequently prevent training injuries.

1. A method for determining a genetic predisposition of a subject to an exercise performance trait, the method comprising:
a) assaying a genetic sample from the subject for a plurality of polymorphisms associated with one or more exercise performance traits selected from exercise endurance, muscular power, muscle damage and/or injury risk, to obtain a polymorphism profile;
b) analysing the polymorphism profile to identify predisposition alleles;
c) assigning a polymorphism score to each polymorphism tested based on the predisposition allele for the associated trait;
d) calculating a total genotype score for each trait, based on a combination of the polymorphism scores for each trait; and
e) classifying the subject's endurance predisposition, power predisposition, muscle damage predisposition and/or injury risk predisposition based on the total genotype score for the one or more exercise performance traits.

2. The method of claim 1, wherein the plurality of polymorphisms associated with exercise endurance is selected from polymorphisms in genes selected from the group consisting of ACE, ACTN3, ADBR2, AMPD1, CKM, EPAS1, GDF-8, HFE, NFATC4, NOS3, NRF2, PPARα, PPARδ, PPARγC1α, PPARγC1β, TFAM, UCP2, UCP3 and VEGFA, but not the combination of genes ACTN3 with ACE when the assaying is for a plurality of polymorphisms associated with exercise endurance only,
wherein the plurality of polymorphisms associated with muscular power exercise is selected from polymorphisms in genes selected from the group consisting of ACE, ACTN3, AMPD1, COL1α1, HIF-1α, IL6, PPARα, PPARγ and VDR,
wherein the plurality of polymorphisms associated with muscle damage is selected from polymorphisms in genes selected from the group consisting of IGF2, IL6, IGF2AS and TNFα, and
wherein the plurality of polymorphisms associated with injury risk is selected from polymorphisms in genes selected from the group consisting of COL1α1, COL5α1 and MMP3.

3-5. (canceled)

6. The method of claim 1, wherein the method comprises assaying a genetic sample from the subject for polymorphisms associated with exercise endurance for at least 2, 3, 4, 5, 6, 7, 8, 9, 10, 11, 12, 13, 14, 15, 16, 17, 18 or 19 genes selected from the group consisting of ACE, ACTN3, ADBR2, AMPD1, CKM, EPAS1, GDF-8, HFE, NFATC4, NOS3, NRF2, PPARα, PPARδ, PPARγC1α, PPARγC1β, TFAM, UCP2, UCP3 and VEGFA, but not the combination of genes ACTN3 with ACE when the assaying is for a plurality of polymorphisms associated with exercise endurance only.

7. The method of claim 1, wherein the method comprises assaying a genetic sample from the subject for polymorphisms associated muscular power exercise for at least 2, 3, 4, 5, 6, 7, 8 or 9 genes selected from the group consisting of ACE, ACTN3, AMPD1, COL1α1, HIF-1α, IL6, PPARα, PPARγ and VDR.

8. The method of claim 1, wherein the method comprises assaying a genetic sample from the subject for polymorphisms associated with muscle damage for at least 2, 3 or 4 genes selected from the group consisting of IGF2, IL6, IGF2AS and TNFα.

9. The method of claim 1, wherein the method comprises assaying a genetic sample from the subject for polymorphisms associated with injury risk for at least 2 or 3 genes selected from the group consisting of COL1α1, COL5α1 and MMP3.

10. The method of claim 1, wherein the polymorphisms are single nucleotide polymorphisms (SNPs).

11. The method of claim 10, wherein the SNPs associated with exercise endurance are selected from the group consisting of SEQ ID NO: 1 (RS4343), or the reverse complement thereof, SEQ ID NO: 2 (RS1815739), or the reverse complement thereof, SEQ ID NO: 3 (RS1042713), or the reverse complement thereof, SEQ ID NO: 4 (RS17602729), or the reverse complement thereof, SEQ ID NO: 5 (RS1803285), or the reverse complement thereof, SEQ ID NO: 8 (RS1867785), or the reverse complement thereof, SEQ ID NO: 9 (RS11689011), or the reverse complement thereof, SEQ ID NO: 10 (RS1805086), or the reverse complement thereof, SEQ ID NO: 11 (RS1799945), or the reverse complement thereof, SEQ ID NO: 18 (RS2229309), or the reverse complement thereof, SEQ ID NO: 19 (RS1799983), or the reverse complement thereof, SEQ ID NO: 20 (RS7181866), or the reverse complement thereof, SEQ ID NO: 21 (RS4253778), or the reverse complement thereof, SEQ ID NO: 22 (RS2016520), or the reverse complement thereof, SEQ ID NO: 24 (RS8192678), or the reverse complement thereof, SEQ ID NO: 25 (RS7732671), or the reverse complement thereof, SEQ ID NO: 26 (RS11959820), or the reverse complement thereof, SEQ ID NO: 27 (RS1937), or the reverse complement thereof, SEQ ID NO: 29 (RS660339), or the reverse complement thereof, SEQ ID NO: 30 (RS1800849), or the reverse complement thereof, and SEQ ID NO: 32 (RS2010963), or the reverse complement thereof,
wherein the SNPs associated with muscular power are selected from the group consisting of SEQ ID NO: 1 (RS4343), or the reverse complement thereof, SEQ ID NO: 2 (RS1815739), or the reverse complement thereof, SEQ ID NO: 4 (RS17602729), or the reverse complement thereof, SEQ ID NO: 6 (RS1800012), or the reverse complement thereof, SEQ ID NO: 12 (RS11549465), or the reverse complement thereof, SEQ ID NO: 15 (RS1800795), or the reverse complement thereof, SEQ ID NO: 21 (RS4253778), or the reverse complement thereof, SEQ ID NO: 23 (RS1801282), or the reverse complement thereof, and SEQ ID NO: 31 (RS2228570), or the reverse complement thereof,
wherein the SNPs associated with muscle damage are selected from the group consisting of SEQ ID NO: 13 (RS3213221), or the reverse complement thereof, SEQ ID NO: 14 (RS680), or the reverse complement thereof, SEQ ID NO: 15 (RS1800795), or the reverse complement thereof, SEQ ID NO: 16 (RS7924316), or the reverse complement thereof, and SEQ ID NO: 28 (RS1800629), or the reverse complement thereof, and
wherein the SNPs associated with injury risk are selected from the group consisting of SEQ ID NO: 6 (RS1800012), or the reverse complement thereof, SEQ ID NO: 7 (RS12722), or the reverse complement thereof, and SEQ ID NO: 17 (RS679620), or the reverse complement thereof.

12-14. (canceled)

15. The method of claim 11, wherein the predisposition allele of SEQ ID NO: 1 (RS4343) is A/A, or the reverse complement thereof, SEQ ID NO: 2 (RS1815739) is T/T, or the reverse complement thereof, SEQ ID NO: 3 (RS1042713) is A/A, or the reverse complement thereof, SEQ ID NO: 4 (RS17602729) is G/G or A/G, or the reverse complements thereof, SEQ ID NO: 5 (RS1803285) is C/T, or the reverse complement thereof, SEQ ID NO: 8 (RS1867785) is G/G, or the reverse complement thereof, SEQ ID NO: 9 (RS11689011) is T/T, or the reverse complement thereof, SEQ ID NO: 10 (RS1805086) is T/T, or the reverse complement thereof, SEQ ID NO: 11 (RS1799945) is C/G, or the reverse complement thereof, SEQ ID NO: 18 (RS2229309) is G/G, or the reverse complement thereof, SEQ ID NO: 19 (RS1799983) is G/G, or the reverse complement thereof, SEQ ID NO: 20 (RS7181866) is G/G, or the reverse complement thereof, SEQ ID NO: 21 (RS4253778) is G/G, or the reverse complement thereof, SEQ ID NO: 22 (RS2016520) is C/C, or the reverse complement thereof, SEQ ID NO: 24 (RS8192678) is C/C, or the reverse complement thereof, SEQ ID NO: 25 (RS7732671) is C/C, or the reverse complement thereof, SEQ ID NO: 26 (RS11959820) is A/A, or the reverse complement thereof, SEQ ID NO: 27 (RS1937) is C/C, or the reverse complement thereof, SEQ ID NO: 29 (RS660339) is A/A, or the reverse complement thereof, SEQ ID NO: 30 (RS1800849) is A/A, or the reverse complement thereof, and/or SEQ ID NO: 32 (RS2010963) is C/C, or the reverse complement thereof are associated with exercise endurance,
wherein the predisposition allele of SEQ ID NO: 1 (RS4343) is G/G, or the reverse complement thereof, SEQ ID NO: 2 (RS1815739) is C/C, or the reverse complement thereof, SEQ ID NO: 4 (RS17602729) is G/G or A/G, or the reverse complements thereof, SEQ ID NO: 6 (RS1800012) is C/C, or the reverse complement thereof, SEQ ID NO: 12 (RS11549465) is C/T, or the reverse complement thereof, SEQ ID NO: 15 (RS1800795) is G/G, or the reverse complement thereof, SEQ ID NO: 21 (RS4253778) is C/C, or the reverse complement thereof, SEQ ID NO: 23 (RS1801282) is G/G or G/C, or the reverse complements thereof, and/or SEQ ID NO: 31 (RS2228570) is T/T, or the reverse complement thereof are associated with muscular power,
wherein the predisposition allele of SEQ ID NO: 13 (RS3213221) is G/G, or the reverse complement thereof, SEQ ID NO: 14 (RS680) is A/A, or the reverse complement thereof, SEQ ID NO: 15 (RS1800795) is C/C or G/C, or the reverse complements thereof, SEQ ID NO: 16 (RS7924316) is T/T, or the reverse complement thereof, and/or SEQ ID NO: 28 (RS1800629) is G/G, or the reverse complement thereof is associated with muscle damage, and
wherein the predisposition allele of SEQ ID NO: 6 (RS1800012) is C/C or A/C, or the reverse complements thereof, SEQ ID NO: 7 (RS12722) is C/T, or the reverse complement thereof, and/or SEQ ID NO: 17 (RS679620) is C/C, or the reverse complement thereof are associated with injury risk.

16-18. (canceled)

19. The method of claim 1, comprising the further step of assaying the genetic sample to determine a haplogroup.

20. The method of claim 19, wherein the step of assaying the genetic sample to determine a haplogroup comprises assaying a mitochondrial polymorphism or a Y-chromosome polymorphism.

21. A method for formulating an exercise program for improving physical performance in a subject, the method comprising:
a) assaying a genetic sample from the subject for a plurality of polymorphisms associated with one or more exercise performance traits selected from exercise endurance, muscular power, muscle damage and/or injury risk, to obtain a polymorphism profile;
b) analysing the polymorphism profile to identify predisposition alleles;
c) assigning a polymorphism score to each polymorphism tested based on the predisposition allele for the associated trait;
d) calculating a total genotype score for each trait, based on a combination of the polymorphism scores for each trait;
e) classifying the subject's endurance predisposition, power predisposition, muscle damage predisposition and/or injury risk predisposition based on the total genotype score for the one or more exercise performance traits; and
f) formulating an exercise plan for the subject, comprising exercises suitable for the subject's predisposition.

22. The method of claim 1, wherein the genetic sample is a buccal sample.

23. The method of claim 1, comprising the further step of counselling the subject.

24. The method of claim 1, comprising the further step of providing the subject with a dietary regimen.

25. The method of claim 24, wherein the dietary regimen comprises specific amounts of macronutrients.

26. (canceled)

27. A kit for identifying a genetic predisposition of a subject to increased exercise endurance, increased muscular power, muscle damage and/or injury risk, the kit comprising a sampler for obtaining a genetic sample from a subject and reagent for assaying a genetic sample obtained from the subject for a plurality of polymorphisms in genes selected from the group consisting of ACE, ACTN3, ADBR2, AMPD1, CKM, COL1α1, COL5α1, EPAS1, GDF-8, HFE, HIF-1α, IGF2, IL6, IGF2AS, MMP3, NFATC4, NOS3, NRF2, PPARα, PPARδ, PPARγC1α, PPARγC1β, PPARγ, TFAM, TNFα, UCP2, UCP3, VDR and VEGFA or combination thereof.

28. (canceled)
